(19) United States
(12) Patent Application Publication
(10) Pub. No.: US 2019/0388181 A1
(43) Pub. Date: Dec. 26, 2019

(54) PRECONDITIONING APPARATUS AND PROCESS FOR ENDOSCOPE REPROCESSING

(71) Applicant: Medivators Inc., Minneapolis, MN (US)

(72) Inventors: Michael P. Petersen, Eden Prairie, MN (US); Nicholas G. Brorrsiley, Annandale, MN (US); David c. Hemink, Minnetonka, MN (US); Joseph G. Johnnie, Pearland, TX (US); Brett Riske, Saint Michael, MN (US); William Haydon, Shorewood, MN (US)

(21) Appl. No.: 16/485,593

(22) PCT Filed: Feb. 16, 2018
(86) PCT No.: PCT/US18/18489

§ 371 Date: Aug. 13, 2019

Related U.S. Application Data
(60) Provisional application No. 62/460,144, filed on Feb. 17, 2017.

(51) Int. Cl. A61B 90/70; B08B 9/043; A46B 9/00; A46B 9/02

(52) U.S. Cl. CPC A61B 90/70; B08B 9/043; A61B 2090/701; A46B 9/026; A46B 9/005

(57) ABSTRACT

The present invention relates to systems and methods for preconditioning endoscopes. In one aspect, the present invention is directed to an endoscope preconditioning system that includes a sealable container to retain a contaminated endoscope, a foamed detergent source, a first system for applying foamed detergent to the outer surface of the endoscope, and a second system for applying foamed detergent or liquid detergent to the interior channels of the endoscope. In some embodiments of this endoscope preconditioning system, foamed or liquid detergent is applied to interior channels of the endoscope with a cleaning device.

### PRIORITY

This application claims priority to U.S. Provisional Patent Application No. 62/460,144 filed Feb. 17, 2017, which is hereby incorporated by reference in its entirety for all purposes.

### TECHNICAL FIELD

The present invention relates to systems and methods for preconditioning soiled endoscopes for reprocessing.

### BACKGROUND

After an endoscope is used on a patient, a number of steps are required to reprocess the endoscope before the contaminated endoscope can be reused. Included in the steps of reprocessing an endoscope is a cleaning and disinfecting step using, for example, a liquid disinfectant solution.

Apparatus and devices designed for, at least partially, automated cleaning and sterilization of medical devices and instruments are known. Automated Endoscope Reprocessors (AERs) are important devices widely used in the health care setting to reprocess lumen-containing medical devices including robotic endoscopic instruments, flexible and rigid type endoscopes, such as duodenoscopes and endoscope accessories, to decontaminate and clean them between uses. AERs are designed to kill microorganisms in or on reusable endoscopes by exposing their outside surfaces and interior channels to chemical solutions.

AER and endoscope manufacturers consider the long narrow channels of endoscopes and duodenoscopes to be particularly challenging areas of the device to reprocess. In addition, the recess around the duodenoscope's elevator area also poses challenges to effectively reprocess duodenoscopes.

Due to the potential association between reprocessed endoscopes and duodenoscopes and patient infection, AER and endoscope manufacturers conduct rigorous validation testing with endoscopes and duodenoscopes to evaluate AER reprocessing effectiveness of the recess around the duodenoscope's elevator area. It is important in these processes to minimize the time between the contamination of the endoscope in the procedure room and the cleaning and disinfection or sterilization of the endoscope. If there is a long period of time, such as more than one hour, the contaminants on the endoscope have time to dry out which makes them much harder to remove. If these contaminants are not removed, it can compromise the high level disinfection or sterilization of the endoscope.

To support adequate high level disinfection or liquid chemical sterilization, testing is expected to demonstrate a 99.9999% reduction of the most resistant microbes at several locations on the endoscope or duodenoscope to prevent infection transmission.

### SUMMARY

There exists a need for apparatus and processes to properly reprocess endoscopes and endoscope accessories to prevent patient contamination and infection.

This disclosure includes representative embodiments of preconditioning apparatus and processes for reprocessing endoscopes including, for example, colonoscopes and duodenoscopes, and endoscope accessories.

In one aspect, the present invention is directed to an endoscope preconditioning system that includes a sealable container to retain a contaminated endoscope, a foamed detergent source, a first system for applying foamed detergent to the outer surface of the endoscope, and a second system for applying foamed detergent or liquid detergent to the interior channels of the endoscope. In some embodiments of this endoscope preconditioning system, foamed detergent is applied to interior channels of the endoscope. In other embodiments, liquid detergent is applied to interior channels of the endoscope.

In this preconditioning system, foam or liquid detergent may be applied to the interior channels in several ways or modes. In some embodiments, foamed or liquid detergent is applied under positive pressure to an endoscope suction valve port or connector, an endoscope air/water valve port or water-jet connector, or an endoscope biopsy channel inlet to add foamed or liquid detergent to an air/water channel, biopsy channel, or biopsy/suction channel of the endoscope. In other embodiments foamed or liquid detergent is applied under positive pressure to the distal tip of the endoscope to add foamed or liquid detergent to an air/water channel, biopsy channel, or biopsy/suction channel of the endoscope. In still other embodiments, foamed detergent is applied at ambient pressure to a distal tip of the endoscope and foamed detergent is drawn into an air/water channel, biopsy channel, or biopsy suction channel of the endoscope by applying negative pressure suction to a biopsy channel inlet, air/water valve port, or suction valve port of the endoscope. In other embodiments, foamed or liquid detergent is applied at ambient pressure to a biopsy channel inlet, air/water valve port, suction valve port, water-jet connector or suction connector of the endoscope and foamed or liquid detergent is drawn into an air/water channel, biopsy channel, air channel, water channel, or biopsy/suction channel of the endoscope by apply negative suction or vacuum to the distal tip of the endoscope, in other embodiments, the channels can be balloon filling channels, balloon evacuation channels, and/or elevator wire channels.

In some embodiments of this endoscope preconditioning system, foamed or liquid detergent is applied to interior channels of the endoscope, and the interior channels of the endoscope are cleaned or treated with a cleaning apparatus or device. In other embodiments, foamed detergent is applied to outer surfaces of the endoscope and is also applied with a cleaning device, such as a brush or a cylindrical member with bristles disposed therein to envelop and clean these outer surfaces.

In another aspect, the present invention is directed to a method of preconditioning an endoscope contaminated with gross bioburden comprising the steps of i) retaining the contaminated endoscope in a container, contacting the outer surfaces of the endoscope with a foamed detergent, iii) contacting the interior channels of the endoscope with the foamed detergent or liquid detergent to provide a pretreated endoscope, and optionally sealing the pretreated endoscope in the container. In some embodiments the sealable container may be a rigid tub or bin, optionally having a rigid or flexible cover. In other embodiments, the sealable container may be a flexible, sealable bag.

In some embodiments, a cleaning device is used to contact the interior channels of the endoscope. In some embodiments, the step of contacting the interior channels of the endoscope further includes laterally moving a cleaning device having an elongate configuration, a distal portion of the elongate configuration including a cleaning member with radially extending members that clean the interior channels of the endoscope. In one example embodiment, the radially extending members include a plurality of disks (rigid or semi-rigid silicone or plastic, for example) or a plurality of bristle members. In related embodiments, the radially extending members are overlaid a portion of the elongate configuration or a cylindrical shaft includes one or more holes or pores configured to distribute the foamed or liquid detergent to the interior channels of the endoscope, the pores or holes facilitating the seepage of the detergent in between the radially extending members.

In yet other related embodiments, the cylindrical shaft includes an elongate cylindrical member disposed on the cylindrical shaft to distribute the foamed or liquid detergent to the interior channels of the endoscope. In these embodiments, the elongate cylindrical member is one of a solid, porous or sponge-like construction. The elongate cylindrical member, in one example embodiment, includes a series of ring members disposed thereon that work like nubs to clean the interior channels of the endoscope.

In embodiments of the endoscope preconditioning system and method, external conduits, tubing, or connectors may be used to provide foamed detergent to the interior volume of the container as well as providing foamed detergent or liquid detergent to interior channels of the endoscope.

In another aspect, the present invention is directed to a flexible bag for preconditioning a contaminated endoscope. The bag comprises a transparent polymeric bag to retain the contaminated endoscope in an interior volume of the bag, connectors to allow materials to escape from the interior of the bag, a connector to deliver foamed detergent to the interior volume of the bag from an external foamed detergent delivery source, and conduits to deliver the foamed detergent to one or more interior channels of the endoscope.

In another aspect, the present invention is directed to a flexible bag for preconditioning a contaminated endoscope comprising a transparent polymeric bag to retain the contaminated endoscope in an interior volume of the bag, a foamed detergent delivery source in the bag, and a connector to remove material from the interior of the bag by applying negative pressure or vacuum to the connector.

In another aspect, the present invention is directed to a flexible bag for preconditioning a contaminated endoscope comprising a transparent polymeric bag to retain the contaminated endoscope in an interior volume of the bag, a foamed detergent delivery source in the bag, and a connector or aperture to allow material from the interior of the bag to vent to the exterior of the bag.

In another aspect, the present invention is directed to a flexible bag for preconditioning a contaminated endoscope comprising a transparent polymeric bag to retain the contaminated endoscope in an interior volume of the bag, a foamed detergent delivery source in the bag, an aperture in the bag to allow material from the interior of the bag to vent to the exterior of the bag, and an aperture cover to seal the bag after foamed detergent is delivered from the foamed detergent delivery source.

In another aspect, the present invention is directed to a flexible bag for preconditioning a contaminated endoscope comprising a semipermeable bag to retain the contaminated endoscope in an interior volume of the bag and allow materials to escape or vent from the interior of the bag, and means to deliver foamed detergent to the interior volume of the bag from an external or internal foamed detergent delivery source.

In another aspect, the present invention is directed to the use of a foamed detergent to precondition a contaminated endoscope. When used, the foamed detergent is stable; immediately breaks down gross bioburden including organic materials, microbes, viruses; encapsulates gross bioburden to prevent drying and hardening of debris; will not slosh or spill; and maintains moisture to prevent adhesion or drying of contaminants.

In all of the various embodiments, conduits may be connected to the interior channels of the endoscope to remove materials from the channels and deliver or draw foamed detergent or liquid detergent into the channels. In other embodiments conduits may be connected to the interior channels of the endoscope to remove materials from the interior channels of the endoscope and draw foamed detergent into the channels when foamed detergent is delivered from a foamed detergent delivery source.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive. All references cited in the instant specification are incorporated by reference for all purposes. Moreover, as the patent and non-patent literature relating to the subject matter disclosed and/or claimed herein is substantial, many relevant references are available to a skilled artisan that will provide further instruction with respect to such subject matter.

### BRIEF DESCRIPTION OF THE DRAWINGS

The invention may take form in various components and arrangements of components, and in various steps and arrangements of steps. The drawings are only for purposes of illustrating specific embodiments and are not to be construed as limiting the invention.

FIG. 1 is a schematic diagram of an endoscope illustrating interior channels and related valves and connectors.

FIG. 2 is a digital image of an embodiment having an endoscope in a tray that is in in a flexible bag fitted with connectors and conduits to provide foamed detergent to the bag and foamed or liquid detergent to the interior channels of the endoscope.

FIG. 3 is a digital image of a connector of a flexible bag connected via a conduit to a vacuum source.

FIGS. 4A and 4B are digital images of a distal end of an endoscope illustrating foamed detergent provided to interior channels of the endoscope.

FIG. 5 is a digital image of an embodiment having an endoscope in a flexible bag fitted with connectors and conduits to provide foamed detergent to the bag and foamed or liquid detergent to the interior channels of the endoscope.

FIG. 6 is a digital image of the internal connections and conduit (tubing) to provide a foamed or liquid detergent to the interior channels of an endoscope.

FIG. 7 is an illustration of an embodiment of a cleaning device for reprocessing an endoscope.

FIGS. 8A-8D illustrate individual example embodiments of cleaning devices for reprocessing an endoscope.

### DETAILED DESCRIPTION

Procedures for reprocessing endoscopes, such as colonoscopes and duodenoscopes, involve a number of critical steps to ensure rigorous cleaning and disinfection of contaminated endoscopes. As part of the reprocessing process, a clinician generally pulls or pushes a detergent solution through the interior channels of the endoscope to start the cleaning process and would then send the endoscope to a manual cleaning sink in a dedicated reprocessing area. It is important in the reprocessing process that the reprocessing begins immediately after the endoscope is used. If the process is delayed, such as having the process delayed for more than an hour after the endoscope is used, accepted reprocessing procedures may require an endoscope to be soaked in a detergent solution for as much as 8 hours before the endoscope then undergoes the rigorous reprocessing process. This soaking procedure makes the endoscope not useable in clinic procedures for an entire day.

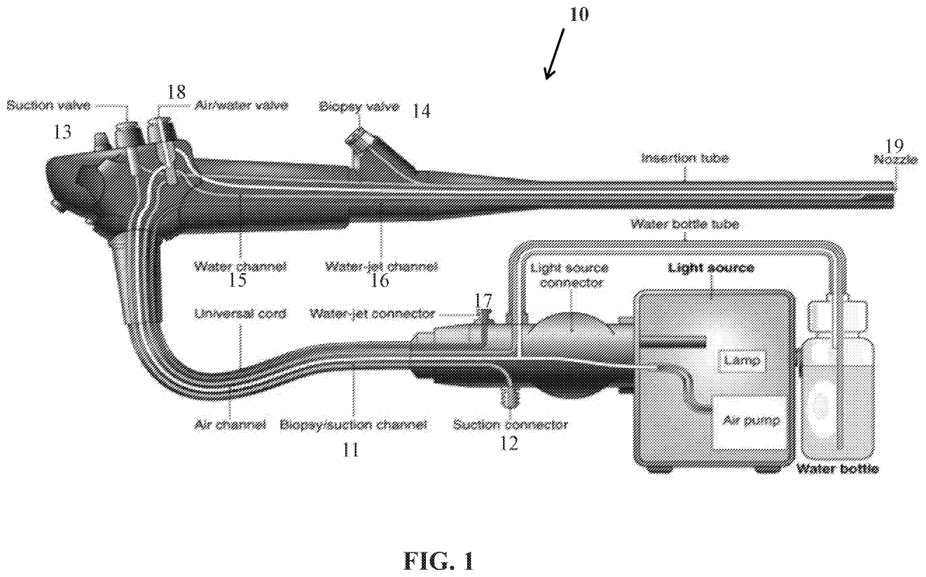

FIG. 1 schematically illustrates the interior channel architecture of an endoscope 10 as well as the related valves and access connectors of the endoscope that allow foamed or liquid detergent to be added to the interior channels during reprocessing. The biopsy/suction channel 11 may be accessed by or connected to a foamed or liquid detergent source through suction connector 12, suction valve port 13 or biopsy channel inlet 14. Water channel 15 and water-jet channel 16 may be accessed by or connected to a foamed or liquid detergent through water-jet connector 17 or air/water valve port 18. Other types of scopes may have additional or different channels, such as balloon filling channels, balloon evacuation channels, and elevator wire channels, which are all within the scope of the present invention.

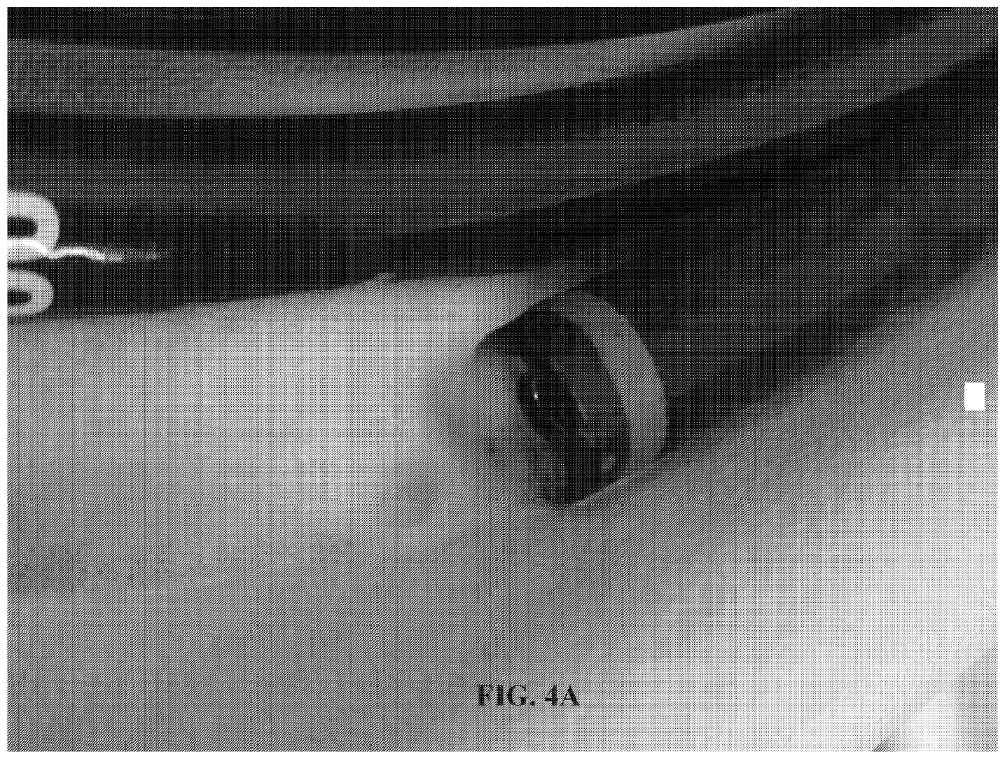

The foamed or liquid detergent may be introduced in the channels in several modes. In one embodiment, foamed or liquid detergent may be introduced into the interior channels 11, 15 and/or 16 via the distal end 19 of the endoscope 10 by forcing the detergent under positive pressure through a connector that fits over the outside of the distal end 19. In this embodiment the appropriate connectors or valves would be opened in order to allow air to escape and the channel to be filled. In another embodiment, foamed or liquid detergent may be drawn into the interior channels 11, 15 and/or 16 by placing the distal end 19 of the endoscope 10 in a source of detergent and then applying negative pressure or vacuum to the appropriate connectors or valves to draw the detergent into the interior channels. In another embodiment, foamed or liquid detergent may be forced into interior channels 11, 15 and/or 16 under positive pressure by connecting a source of detergent to the appropriate connector or valve and forcing the detergent through the interior channels to the distal end 19 of the endoscope 10. In still another embodiment; a negative pressure or vacuum may be applied to the distal end 19 of the endoscope 10 through a connector that fits over the outside of the distal end 19. In this embodiment, the appropriate connectors or valves may be connected to a suitable detergent source so that the detergent may be drawn through the interior channels 11, 15 and/or 16 to the distal end 19 when negative pressure or vacuum is applied to the distal end 19 of the endoscope 10. One skilled in the art would readily appreciate that a variety of connectors, conduits and tubing would be suitable for applying the foamed detergent to the interior channels of the endoscope illustrated in FIG. 1. FIGS. 4A and 4B illustrate a foamed detergent added to the interior channels of the endoscope and exiting the distal end of the interior channels.

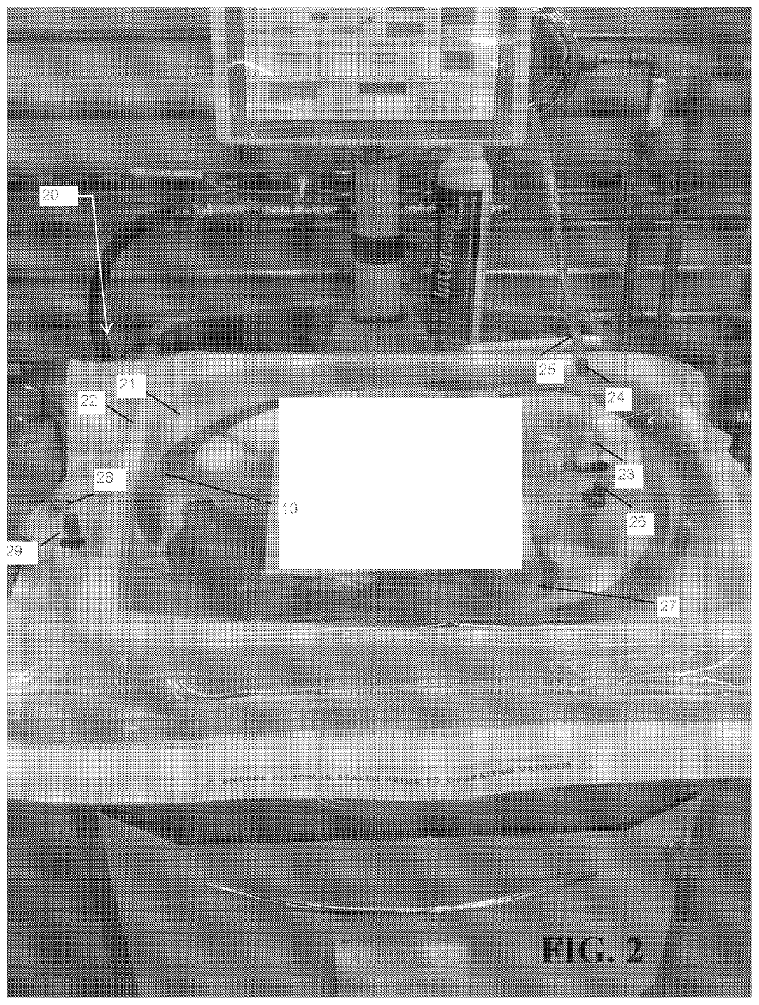

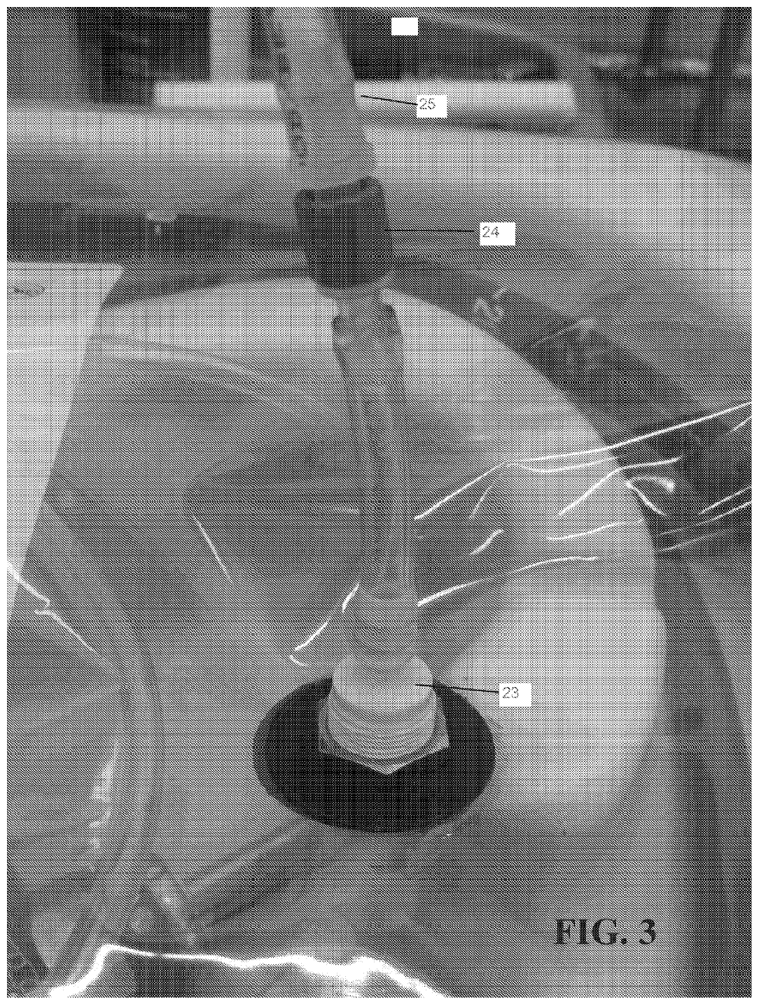

FIG. 2 illustrates an embodiment of a preconditioning system of the present disclosure. In this embodiment, a contaminated endoscope 10, such as a colonoscope, is placed in a cleaning tray 21 and the endoscope and tray are then placed in a transparent, flexible bag or pouch 22. The bag may be sealed or closed so that negative pressure or vacuum can be applied to the bag. The vacuum in FIG. 2 is applied to the bag through connectors 23 and 24. These vacuum connectors 23 and 24 are also illustrated in FIG. 3. The connectors in FIG. 3 include fittings that allow the bag to be connector to a vacuum pump through a suitable tubing 25.

FIG. 2 also illustrates additional connectors and conduits that can be used to provide foamed detergent into the bag to contact the outside surfaces of the endoscope 10 as well as provide foamed or liquid detergent to the interior channels of the endoscope. In FIG. 2, connector 26 is connected to the connectors or the valve ports of the endoscope using suitable tubing segments and additional fittings 27. Additional connectors 28 and 29 allow for foamed detergent to be added to the interior of the bag or to allow fluids or materials to be removed from the bag.

There are alternative embodiments to the embodiment illustrated in FIG. 2. For example, the endoscope 10 may be placed in several different types of container. In one embodiment, the endoscope 10 may be placed in a sealable bag or a pouch that is an opaque, translucent, or transparent polymer film or sheet. In other embodiments, the bag or pouch may be a semipermeable polymer to allow air or other materials to be released or removed from the interior of the bag so that one or more of the connectors illustrated in FIG. 2 may be eliminated. In other embodiments, the bag or pouch may have a sealable aperture or opening in the bag to allow air or other materials to be released or removed from the interior of the bag so that one of more of the connectors illustrated in FIG. 2 may be eliminated. The aperture or opening in the bag may be suitably sealed with, for example, a pressure sensitive film or tape. In still other embodiments, the container may be a tray made from an opaque, translucent, or transparent polymer having a sealable cover made from the same polymer, or from a different polymer, as the polymer of the tray. In still other embodiments, one skilled in the art would readily appreciate that a variety of connectors, conduits and tubing would be suitable for use and could be used in place of the connectors, conduits and tubing in the embodiment illustrated in FIG. 2.

One embodiment of this disclosure is when the bag illustrated in FIG. 2 is used in a preconditioning process. In this embodiment, a contaminated endoscope is connected to the bag's connectors and tubing so the when the bag connector is connected to an external pressurized foamed detergent source, the foamed detergent may be provided to and through the connector and valves of the endoscope so that the foamed detergent flows through the internal channels to the distal end of the endoscope and then exits the distal end and fills the internal volume of the bag. In order to facilitate the filling process, one or more open connectors or apertures would allow materials to escape or vent from the interior volume of the bag. In one embodiment, when the external surfaces of the endoscope are covered with the foamed detergent the addition of foamed detergent is stopped and the open connectors or aperture are closed or sealed and the endoscope may be contained in the bag until rigorous reprocessing can be done. Because all the internal and external contacting surfaces of the endoscope remain damp with the added foam detergent the contacting surfaces never dry out so there is no need for a 8 hour soaking delay in the event there is a delayed period of time between the use of the endoscope in a clinical procedure and subsequent reprocessing of the endoscope.

Another embodiment of this disclosure is when the bag illustrated in FIG. 2 is used in an alternate preconditioning process. In this embodiment, a contaminated endoscope is connected to the bag's connectors and tubing so that when these connectors are connected to an external negative pressure or vacuum the foamed detergent added to the internal volume of the bag will be drawn into the interior channels of the endoscope via the distal end of the scope and then exit the interior channels via the bag's connectors and tubing. For this embodiment, the endoscope's appropriate connectors and valves are connected to the bag's appropriate connectors and then the connected endoscope is place in the bag and the bag is sealed. Foamed detergent is added to the interior volume of the bag either from an internal source of foamed detergent that is placed in the bag before the bag is sealed and then released into the bag after the bag is sealed, or foamed detergent from an external source of foamed detergent is added to the bag when the contaminated endoscope is placed in the bag and the bag is then sealed, or foamed detergent is added to the bag after the bag is sealed via an appropriate bag connector. When the bag has been filled with foamed detergent and sealed, negative pressure or vacuum may be applied to the appropriate bag connectors to draw foamed detergent into the interior channels of the endoscope through the distal end of the endoscope. Because all of the internal and external contacting surfaces of the endoscope remain damp with the added foam detergent the contacting surfaces never dry out so there is no need for a 8 hour soaking delay in the event the is a delayed period of time between the use of the endoscope in a clinical procedure and subsequent reprocessing of the endoscope.

In addition to the embodiments described above, other embodiments of a preconditioning process may be used with a flexible bag, foamed detergent, bag connectors and optional liquid detergent. In one embodiment, a contaminated endoscope may be place in a bag, for example, and the outside surfaces of the endoscope may be completely covered with a foamed detergent from internal or external sources before or after the bag is sealed. During this same general procedure, the interior channels of the endoscope may be with filled with liquid detergent, again, before or after the bag is sealed. The order of the procedure may be varied as needed in order to assure the outer surfaces and interior channels of the endoscope are in contact with the foamed or liquid detergent so that the surfaces will remain in a wet or damp condition in the sealed bag until the endoscope may be fully reprocessed before it next use.

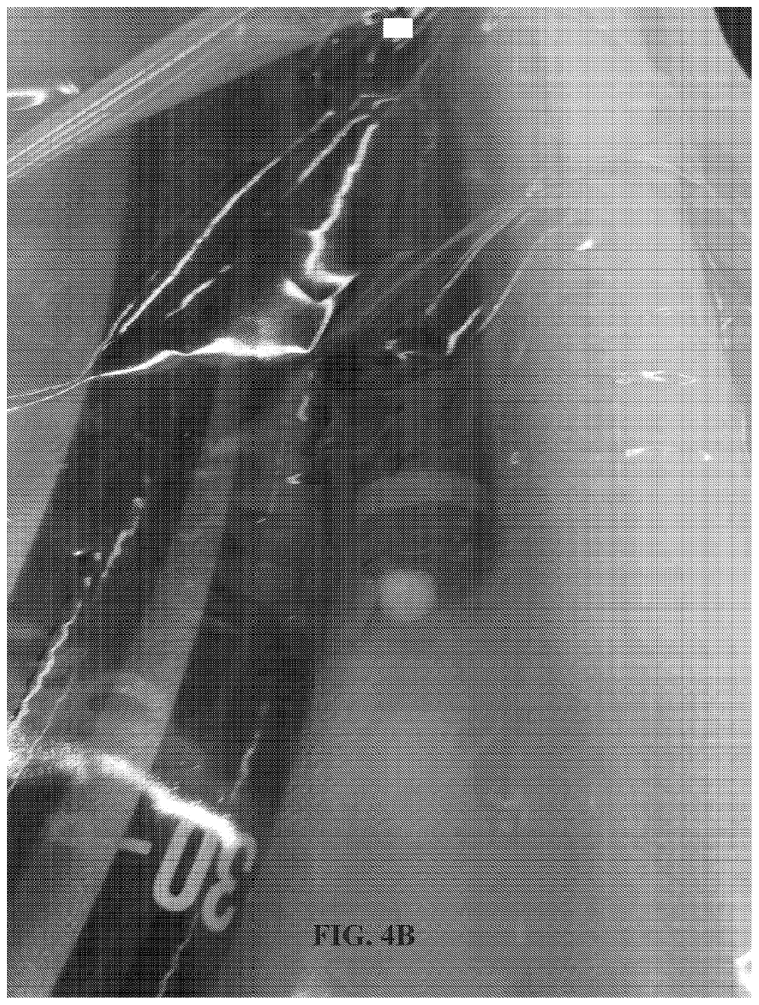

FIG. 5 illustrates an embodiment of a preconditioning system of the present disclosure. In this embodiment, a contaminated endoscope 50 is placed in a transparent, flexible bag or pouch 52. The bag may be sealed or closed so that negative pressure or vacuum can be applied to the bag using a bag sealing fastener 54 along one edge of the bag and a connector or valve to a vacuum source. FIG. 5 also illustrates the bag's external connectors or valves that allow air to escape from the bag, connector 55, allow foam or liquid detergent to be added to the interior channels of the endoscope, connector 56, and allow the foam or liquid detergent to be removed or drained from the bag during the reprocessing procedure, connector 57.

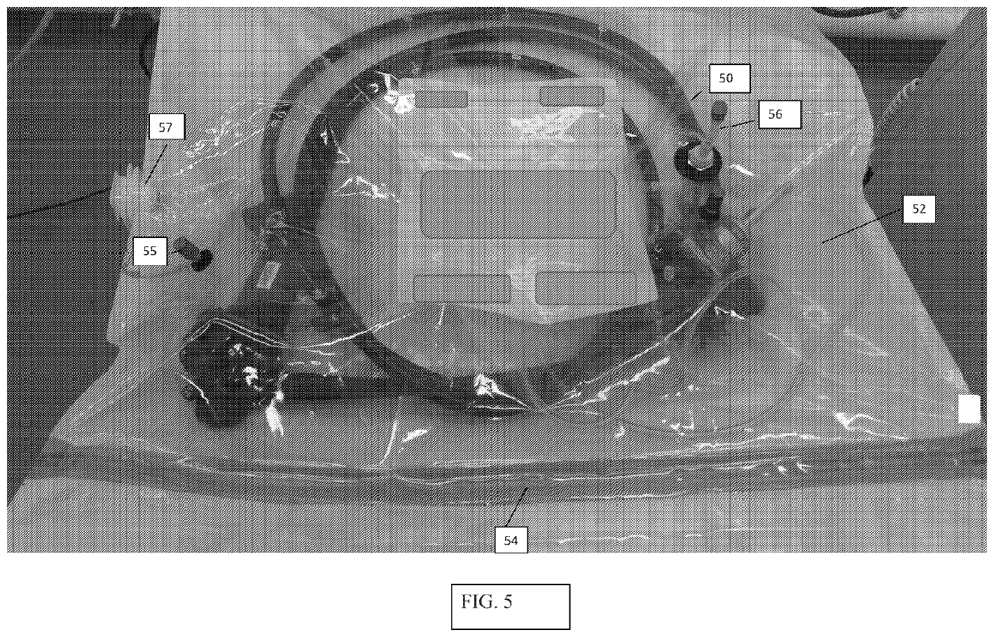

FIG. 6 illustrates an embodiment of the disclosure having connectors, conduits (tubing), and plugs to allow foam or liquid detergent to be added to the system in order to clean, flush and/or sanitize the interior channels of an endoscope. In this embodiment, contaminated endoscope 60 is placed in a transparent, flexible bag 62. The hag includes a vent plug 64 that allows air to vent from the bag as needed during the reprocessing procedure, and also a vacuum source to be connected to the bag. The bag also includes hookup connection 66 that allows foam or liquid detergent to be added to the endoscope connection conduits 68 (clear tubing illustrated in this figure). The conduits are connected to the interior channels of the endoscope at air connection 71, suction connection 72, and elevator connection 73. Also illustrated is channel plug 70.

A suitable foamed detergent for use in the disclosed embodiments is INTERCEPT foam available from Medivators, Plymouth, Minn. A suitable liquid detergent for use in the disclosed embodiments is INTERCEPT detergent also available from Medivators, Plymouth, Minn.

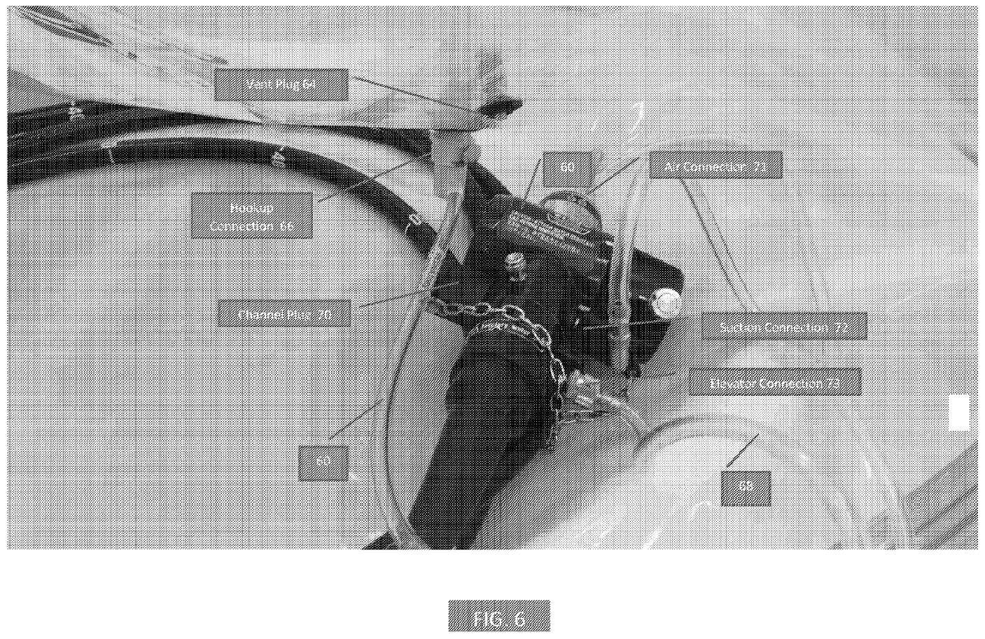

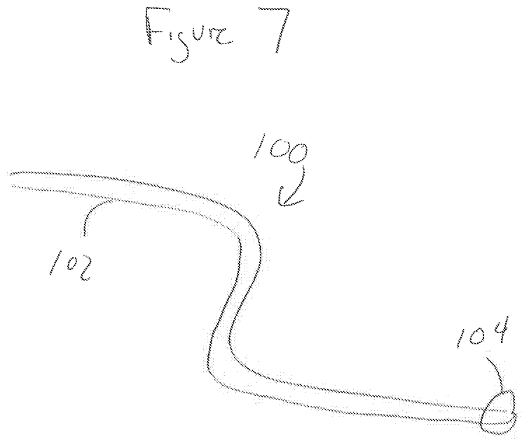

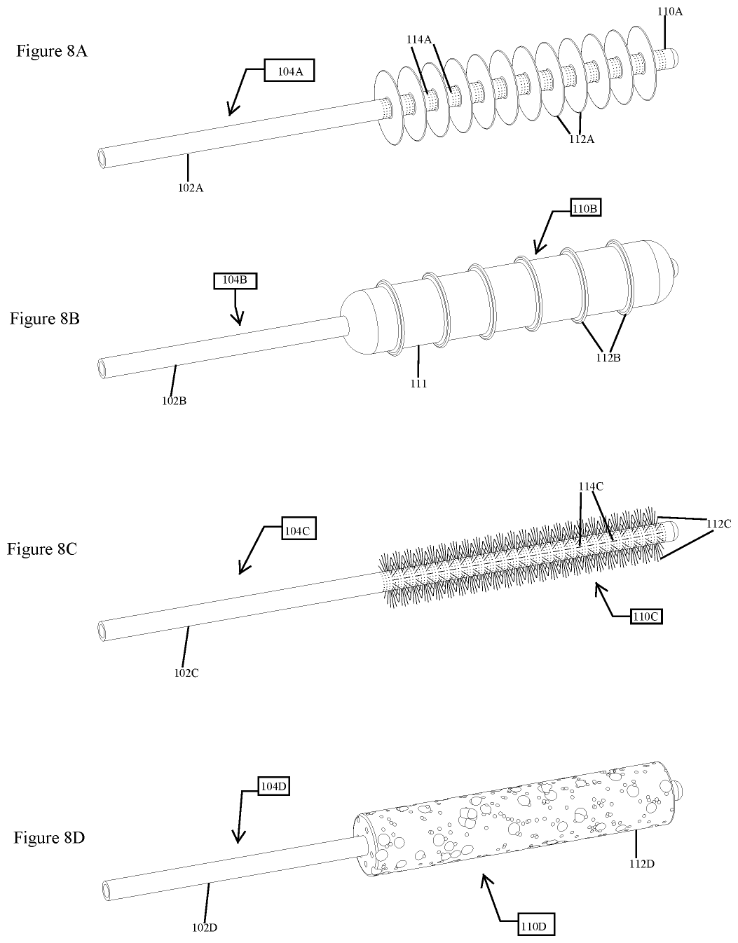

Referring now to FIGS. 7 and 8A-8D, FIG. 7 illustrates an example embodiment of a cleaning device 100. The cleaning device 100 allows the interior channels of the endoscope to be mechanically cleaned of debris and unwanted residue that contaminate the channels. In one embodiment, cleaning device 100 is an applicator system for applying a foam or liquid detergent that generally comprises an elongate shaft 102 and a cleaning end 104. In an embodiment, elongate shaft 102 may be rigid, while in an alternative embodiment, elongate shaft 102 is flexible. Cleaning device 100 may be reusable or may be disposable.

In one example embodiment, FIG. 8A illustrates a cleaning end 104A having a shaft 102A with a cleaning head member 110A formed from one or more disks 112A configured for cleaning the inside channel of the endoscope. In various example embodiments, each of the disks is rigid or semi-rigid to provide for disk deformation within the endoscope channel. In this example embodiment, a portion of shaft 102A which supports one or more disks 112A, includes one or more holes or pores 114 that allow the cleaning foam or liquid detergent to seep through to and around disks 112A.

In another example embodiment, FIG. 8B illustrates a cleaning end 104B having a shaft 102B with a cleaning head member 110B formed from an elongate cylinder 111 disposed about shaft 102B that includes one or more rings 112B configured for cleaning an inside channel of the endoscope. In this example embodiment, cylindrical member 111 is solid and in related embodiments the cylindrical member is formed from a porous or semi-porous material (or one that has a plurality of pores or holes in the cylinder body) to allow for the cleaning solution or foam to seep up or flow out through to cleaning rings 112B.

In another example embodiment, FIG. 8C illustrates a cleaning end 104C having a shaft 102C with a cleaning head member 1100 formed from a plurality of bristles 1120 extending outwardly and perpendicularly from shaft 1020 configured for cleaning the inside channel of the endoscope. The length of each of the plurality of bristles 112C depends on an inner diameter of the endoscopic channel being cleaned.

In yet another example embodiment, FIG. 8D illustrates a cleaning end 104D having a shaft 102D with a cleaning head member 110D formed from a sponge or fibrous cylindrical member 112D configured for cleaning the inside channel of the endoscope. In a related embodiment, fibrous member 112D includes either rings (as in rings 112B) or include disks 112A that surround (and are disposed thereon) a thinner cylindrical member 112D to allow the cleaning solution or foam to seep up through to cleaning rings or disks.

Cleaning end 104 may include a plurality of extending members 110, wherein each extending member 110 extends outward from elongate shaft 102, for example at a perpendicular angle. In an embodiment, the extending members 110 are fixed, while in another embodiment, the extending members 110 are expandable. Extending members 110 may be bristle-like, comprising a material selected from polyester, polypropylene, cotton, woven fabrics, non-woven fabrics, nylon; or polyamide derivatives.

Alternatively, extending members 110 may be located along the length of the elongate shaft 102.

One embodiments of a cleaning end 104 may include an expandable squeegee. Such a device is capable of expanding in diameter to be used in endoscopes having interior channels with different diameter. A squeegee may be made from materials selected from flexible plastics, rubber, or similar materials.

Another cleaning end 104 may comprise a balloon catheter-type expanding member. In various example embodiments, the balloon portion disposed on the shaft of the cleaning device is: 1) formed of different sizes and includes various folds to accommodate balloon expansion; 2) one or more balloons are disposed along the shaft of cleaning device; and 3) the surface of the balloon and the folds include nubs or bumps to assist in cleaning the interior channels of the endoscope when the balloon is expanded and is combined with the cleaning foam or liquid detergent. In a related embodiment, the balloon or balloons disposed about the shaft of the cleaning device (as well as the shaft) include pores or holes to allow the foam or liquid detergent to flow through the shaft and into the balloon and fill the it with detergent that thereafter seeps from the balloon as it is cleaning the interior diameter and surfaces of the channels.

Modifications and variation of the above-described embodiments of the invention are possible without departing from the invention, as appreciated by those skilled in the art in light of the above teachings. It is therefore understood that, within the scope of the claims and equivalents, the invention may be practiced otherwise than as specifically described.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention.

1. An endoscope preconditioning system comprising a sealable container to retain a contaminated endoscope having an outer surface and interior channels, a foamed detergent source, a first system for applying foamed detergent to the outer surface of the endoscope, and a second system for applying foamed detergent or liquid detergent to the interior channels of the endoscope.

2. The endoscope preconditioning system of claim 1, wherein the interior channels of the endoscope contain foamed detergent.

3. The endoscope preconditioning system of claim 1, wherein the interior channels of the endoscope contain liquid detergent.

4. The endoscope preconditioning system of claim 1, wherein the second system for applying foamed or liquid detergent to the interior channels of the endoscope is configured to apply the detergent under positive pressure to an endoscope suction valve port or connector, an endoscope air/water valve port or water-jet connector, or an endoscope biopsy channel inlet of the endoscope to add the foamed or liquid detergent to the air/water channel, biopsy channel, or biopsy/suction channel of the endoscope.

5. The endoscope preconditioning system of claim 1, wherein the second system for applying foamed or liquid detergent to the interior channels of the endoscope is configured to apply the detergent under positive pressure to a distal tip of the endoscope to add foamed or liquid detergent to an air/water channel, a biopsy channel, or a biopsy/suction channel of the endoscope.

6. The endoscope preconditioning system of claim 1, wherein the second system for applying foamed or liquid detergent to the interior channels of the endoscope is configured to apply the detergent at ambient pressure to a distal tip of the endoscope and wherein the foamed or liquid detergent is drawn into an air/water channel, a biopsy channel, or a biopsy/suction channel of the endoscope by applying negative pressure suction to a biopsy channel inlet, an air/water valve port, or a suction valve port of the endoscope.

7. The endoscope preconditioning system of claim 1, wherein the second system for applying foamed or liquid detergent to the interior channels of the endoscope is configured to apply the detergent at ambient pressure to a distal tip, a suction connector, or a water-jet connector of the endoscope and wherein the foamed or liquid detergent is drawn into an air/water channel, a biopsy channel, an air channel, a water channel, or a biopsy/suction channel of the endoscope by applying negative suction to a biopsy channel inlet, an air/water valve port, a suction valve port, a water-jet connector or a suction connector of the endoscope.

8. The endoscope preconditioning system of claim 1, further comprising a cleaning device.

9. The endoscope preconditioning system of claim 8 wherein the cleaning device comprises a cylindrical shaft configured to receive and distribute the foamed or liquid detergent to the interior channels of the endoscope.

10. The endoscope preconditioning system of claim 9, wherein the cylindrical shaft includes one or more holes or pores configured to distribute the foamed or liquid detergent to the interior channels of the endoscope.

11-15. (canceled)

16. The endoscope preconditioning system of claim 1, wherein the second system comprises conduits, tubing, or connectors configured to provide foamed detergent or liquid detergent to the interior channels of the endoscope.

17. The endoscope preconditioning system of claim 1, wherein the container comprises a rigid tub or bin, optionally having a rigid or flexible cover.

18. (canceled)

19. The endoscope preconditioning system of claim 1, wherein the container comprises a flexible, sealable bag.

20. The endoscope preconditioning system of claim 19, wherein the flexible, sealable bag is a transparent polymeric material.

21. A method of preconditioning an endoscope contaminated with gross bioburden comprising the steps of i) retaining the contaminated endoscope in a container, ii) contacting an outer surface of the endo scope with a foamed detergent, iii) contacting an interior channel of the endoscope with foamed or liquid detergent to provide a pretreated endoscope, and optionally sealing the pretreated endoscope in the container.

22. The method of claim 21, further comprising the step of contacting the interior channels of the endoscope with a cleaning device.

23. The method of claim 22 wherein the step of contacting the interior channels of the endoscope further includes laterally moving the cleaning device having an elongate configuration.

24-32. (canceled)

33. The method of any of claim 21, wherein the container comprises a flexible, sealable bag.

34. The method of claim 33, wherein the flexible, sealable bag is a transparent polymeric material.

35. (canceled)

36. A flexible bag for preconditioning a contaminated endoscope, the flexible bag comprising a transparent polymeric bag to retain the contaminated endoscope in an interior volume of the bag, valves to allow materials to escape from the interior of the bag, a connector to deliver foamed detergent to the interior volume of the bag from an external foamed detergent delivery source, and conduits to deliver foamed or liquid detergent to one or more interior channels of the endoscope.

37-43. (canceled)
